(19) United States
(12) Patent Application Publication
(10) Pub. No.: US 2008/0065072 A1
(43) Pub. Date: Mar. 13, 2008

(54) Kit for Implant Retrieval and Method of Use

(76) Inventors: James Spitler, Taunton, MA (US); Michael S. Castro, Uxbridge, MA (US); Rex Shores, Norfolk, MA (US); Kevin Tahmoush, Hopedale, MA (US); Stefan Gabriel, Mattapoisett, MA (US)

Correspondence Address: CARR LLP (IST), 670 FOUNDERS SQUARE, 900 JACKSON STREET, DALLAS, TX, 75202, US

(21) Appl. No.: 11/852,668

(22) Filed: Sep. 10, 2007

Related U.S. Application Data
(60) Provisional application No. 60/825,082, filed on Sep. 8, 2006.
(60) Provisional application No. 60/825,697, filed on Sep. 14, 2006.
(60) Provisional application No. 60/825,699, filed on Sep. 14, 2006.
(60) Provisional application No. 60/826,789, filed on Sep. 25, 2006.

(51) Int. Cl. A61B 17/70

(52) U.S. Cl. 606/61

(57) ABSTRACT

Disclosed is a kit for use during stabilization procedures. The kit may include a head stabilizer having an elongated shaft with an implant engaging portion coupled to the distal end portion. A holding apparatus may also be included in the kit. The holding apparatus may have a sleeve, a pusher shaft received within the sleeve, and an implant engaging mechanism coupled to the distal end portion of the sleeve. The kit may also include a retrieval tube having a handle portion, an implant engaging mechanism coupled to the distal end portion of the handle portion, and a locking mechanism coupled to the proximal end portion of the handle portion. Other instruments that may be included in the kit are guide wires, dilators, anchors, poly-axial heads, extensions, spinal rods, and set screws.

### CROSS REFERENCE TO RELATED APPLICATIONS

This application claims priority from U.S. provisional patent application Ser. No. 60/825,082 entitled “Implant Holding Apparatus and Method of Implant Retrieval”, filed Sep. 8, 2006, provisional patent application Ser. No. 60/825,697 entitled “Retrieval Tube and Method of Use”, filed Sep. 14, 2006, U.S. provisional patent application Ser. No. 60/825,699 entitled “Head Stabilizer and Method of Poly-axial Head Retrieval”, filed Sep. 14, 2006, and U.S. provisional patent application Ser. No. 60/826,789 entitled “Descriptive Markings for Positive Identification of Instrument Position”, filed Sep. 25, 2006, the disclosure of which is incorporated herein by reference, all of which are herein incorporated by reference

### TECHNICAL FIELD

This application relates generally to the field of implanting medical implant devices and more particularly to systems and methods for inserting bone anchoring devices.

### BACKGROUND INFORMATION

Orthopedic injuries, deformities, and degenerative disease often require intervention in the form of surgery for placing implants to stabilize an internal structure, promote healing and relieve pain. In the area of spinal surgery, for example, a common procedure includes placement of pedicle screws that are joined by a connecting rod spanning between the pedicle screws.

During installation of a pedicle screw an extension is attached to the poly-axial head associated with each pedicle screw. The extension allows access to the pedicle screw once it is in place. The extensions are left in place until the connecting rod is secured between the pedicle screws. Such a procedure is more fully described in a co-pending and commonly assigned U.S. patent application Ser. No. 10/989,715 filed on Nov. 16, 2004 entitled “An Extension for Use with Stabilization Systems for Internal Structures” (hereafter the '715 patent application), which is hereby incorporated by reference.

One problem when connecting the rods to the pedicle screws is that the extensions may become disconnected from the poly-axial heads prematurely. A surgeon must then reconnect the extension to the poly-axial head that is not in a locked or fixed position. Since the poly-axial head is not in a locked or fixed position, the surgeon must attempt to re-establish the connection, without doing more damage to the surrounding tissue and muscle of the patient.

What is needed, therefore, is a device and method to stabilize the poly-axial head while the extension is reattached to the poly-axial head while minimizing the damage to the surrounding tissue and muscle of the patient.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIG. 1 is a perspective view of a kit for retrieving instruments or implants during a surgical procedure.

FIG. 2 illustrates one step in an illustrative embodiment of a procedure for implanting a stabilization system.

FIG. 3 illustrates one step in an illustrative embodiment of a procedure for implanting a stabilization system.

FIG. 4 is a perspective view of an illustrative embodiment of an extension assembly in accordance with the present invention.

FIG. 5 is a perspective view of an illustrative alternative embodiment of an extension assembly in accordance with the present invention.

FIG. 6 is a perspective view of an embodiment of a stabilization system in accordance with the present invention

FIG. 7 is a perspective view of one embodiment of a medical implant holding apparatus incorporating various aspects of the present invention

FIG. 8A is a sectional view of the proximal end of the medical implant holding apparatus of FIG. 7 in an unactuated state;

FIG. 8B is a sectional view of the proximal end of the medical implant holding apparatus of FIG. 7 in an actuated state.

FIG. 9A is a sectional view of the distal end of the medical implant holding apparatus of FIG. 7 in an unactuated state

FIG. 9B is a sectional view of the distal end of the medical implant holding apparatus of FIG. 7 in an actuated state.

FIG. 10 is a flowchart illustrating the steps of using the device of FIG. 7.

FIG. 11 is a perspective view illustrating one embodiment of a retrieval tube in a closed position which incorporates one or more aspects of the invention.

FIG. 12 is a perspective view illustrating one embodiment of a retrieval tube in an open position which incorporates one or more aspects of the invention.

FIG. 13 is a flowchart illustrating the steps of using the device of FIG. 11.

FIG. 14 is a perspective view illustrating one embodiment of a head stabilizer which incorporates one or more aspects of the present invention.

FIG. 15 is an exploded view of an embodiment of the handle interface portion which incorporates one or more aspects of the present invention.

FIG. 16 is an exploded view of one embodiment of an implant engaging mechanism incorporating one or more aspects of the present invention.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

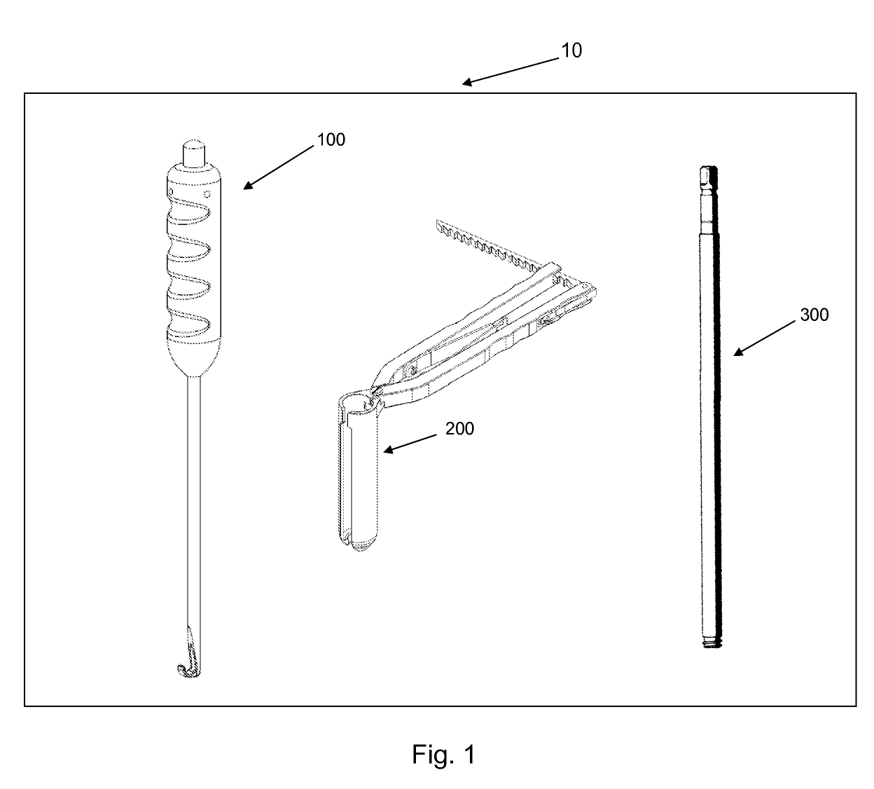

Turning now to FIG. 1, there is presented a kit 10 illustrating a possible embodiment of the present invention. The kit may include a set of instruments for retrieving a poly-axial head that has been prematurely detached from an extension during a minimally invasive surgical procedure. The instruments may include a holding apparatus 100, a retrieval tube 200, and a head stabilizer 300. These devices are described in detail below. Other instruments and implants that may be included in the kit include guide wires, dilators, anchors, poly-axial heads, extensions, spinal rods, and set screws. Different sizes of the devices in the kit may be included. For example, the head stabilizer 300 may include multiple sizes to accommodate a variety of procedures. Details of these instruments may be found in previously incorporated by reference application Ser. No. 10/989,715.

The illustrated instruments of kit 10 may be used during a minimally invasive procedure during which an implant has been detached from an instrument prematurely. The holding apparatus 100, retrieval tube 200, and head stabilizer 300 all may be used to retrieve the detached implant and also may assist in reattaching the implant to the instrument so that the minimally invasive procedure may continue and continue without causing more damage to the tissue surrounding the surgical site.

The manner of using certain aspects of a minimally invasive surgical system to install a pedicle screw system in the spine will now be described. The surgeon identifies the desired vertebral levels and pedicle positions via standard techniques. Once the target vertebrae are identified, a small incision is made through the skin and a tracking needle (or other device) is inserted to pinpoint exactly where each anchor is to be placed. A fluoroscope, or other x-ray technique, may be used to properly position the tracking needle. Once the proper position is located, the guide wire may be positioned with its distal end against the pedicle.

The surgeon may then slide a series of continuing larger sized dilators down the guide wire. Approximately four or five dilators are used until a diameter suitable for passing the anchor and its extensions is achieved. In some embodiments, the last dilator used will be a slotted side dilator. Once the slotted dilator is in place, the other dilators may be removed. In some embodiments, a bone awl and/or bone tap may inserted over the guide wire to tap a hole into the pedicle in preparation for receiving the anchor, which in this case may be a pedicle screw. This tap will usually be a size slightly smaller than the pedicle screw thread size selected for that patient and that level.

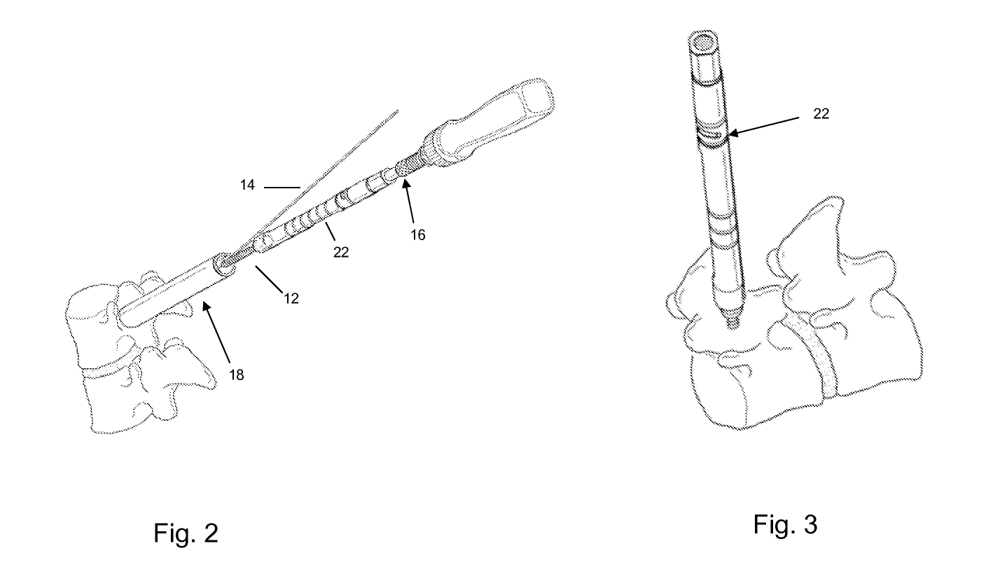

Turning now to FIG. 2, after the hole is tapped and the inner dilators are removed, the surgeon is ready to introduce the anchor into the vertebrae. Prior to inserting the anchor 12 (e.g., pedicle screw), the guide wire 14 is placed through an off-axis bore (not shown) of the anchor 12. The anchor 12 may be coupled to a driving device 16. The driving device 16 engages the proximal end of the anchor 12. As the anchor 12 and the distal end of the driving device 16 enter the slotted dilator 18, the slot of the dilator 18 allows the guide wire 14 to extend beyond the passage of the dilator 18.

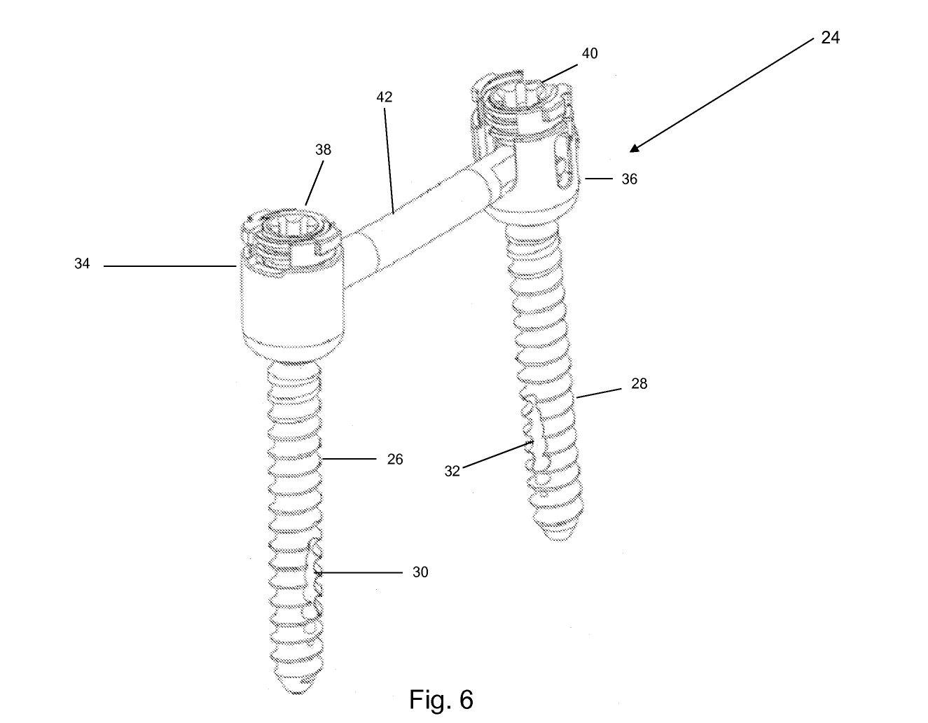

Once the anchor 12 is in position, which may be verified by fluoroscopy techniques, the guide wire 14 may be removed. It may also be desirable at this stage to also remove the dilator 18. Once the anchor 12 is in position, the driving device 16 may then be rotated into a proper position. The surgeon may then screw the anchor 12 into the pre-tapped hole in vertebrae L4. Pressure on the driving device 16 forces the anchor to be in-line with an extension 22 coupled to the anchor 12. The extension 22 may remain coupled to the anchor 12 until the complete pedicle screw system (FIG. 6) is implanted. A similar procedure may be repeated for each additional level, in this case level L5.

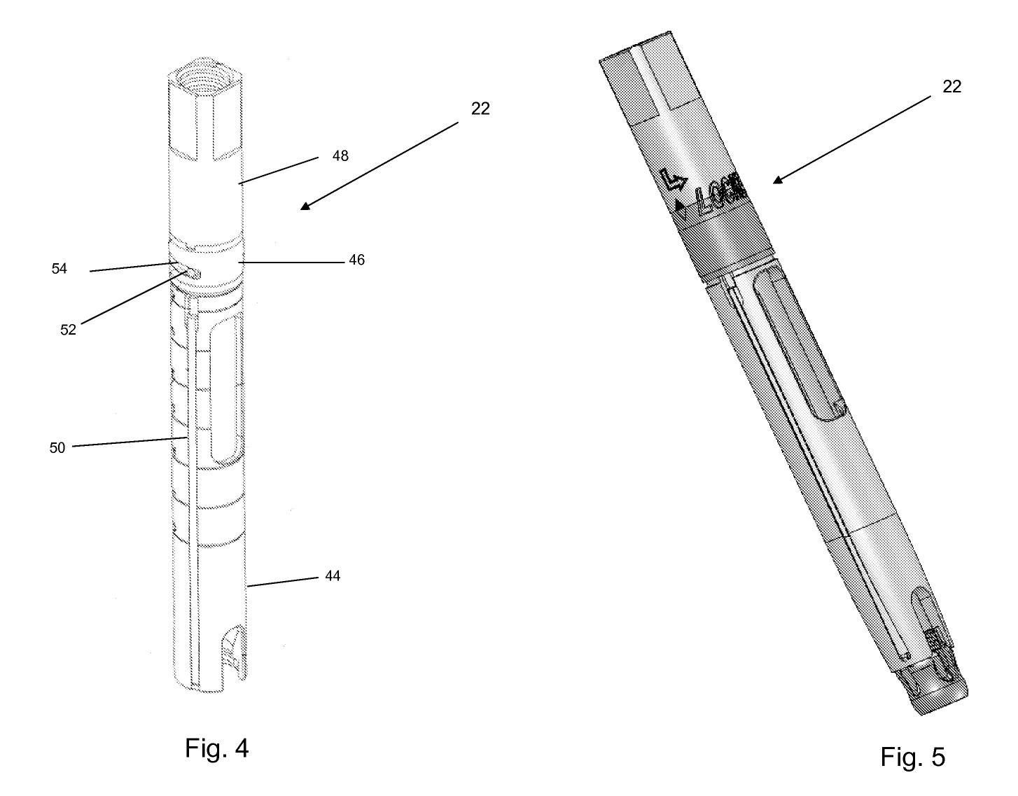

Referring to FIG. 4, a detailed view of one possible embodiment of extension 22 is shown. Extension 22 includes a tube 44 which attaches at one end to an assembly, such as a poly-axial head. Over the opposing end of tube 44 a locking ring 46 is installed. Drive head 48 is used to tighten the extension to a poly-axial head and to provide attachment for an anti torque handle. The drive head 48 attaches to locking ring 46 and tube 44 using a torque key (not shown) for proper positioning. Extension 22 also includes slide 50 which fits into a slot on tube 44 and engages locking ring 46 by means of pin 52.

The locking ring 46 provides the mechanism for locking the extension to the poly-axial head assembly. Locking ring 46 includes a slot 54 which is formed in locking ring at an angle by having the slot begin at one end below the midline of the locking ring and end at the other end above the midline. Slide 52 is coupled to slot 54 of locking ring 46 by means of pin 52 and extends down tube 44 where it can engage with a poly-axial head connected to the extension. The slide 50 locks a poly-axial head with the extension 22. It accomplishes this by sliding up and down the tube 44 in response to the twisting of the locking ring 46. Twisting locking ring 46 causes slot 54 to move from its low end to its high end or vise versa. Pin 52 coupled to slot 54 translates the twisting motion of the locking ring 46 into a linear up and down motion by slide 50 as pin 52 traverses slot 54 from low to high or high to low. A locking extension at the end of slide 50 proximal to the poly-axial head, locks the poly-axial head in place by engaging with slots of the poly-axial head assembly. The poly-axial head is unlocked from the extension 22 by moving the locking extension of slide 50 out of the referenced slots by twisting locking ring 46 such that pin 52 moves to the high position in slot 54.

Turning now to FIG. 5, an alternative embodiment of an extension 22 is shown. The alternative embodiment is similar to the embodiment of FIG. 4. A rotating ring positions a slider bar down to mate within a slot on the implant component or pedicle screw assembly, thereby locking the implant onto the instrument. In this alternative embodiment, a user may want to determine whether the instrument and implant are locked or unlocked. In order to help a user determine the condition of the instrument and implant, the rotating ring of the instrument is marked with the lower portion of the letters of the mark “LOCKED” and the upper body portion of the instrument that is adjacent the proximal end of the ring has the upper portion of the mark “LOCKED”. The instrument is in the locked position when the upper portion and the ring are aligned to clearly read the mark “LOCKED”. FIG. 5 clearly shows the instrument is in the locked position, since the mark “LOCKED” is clearly readable by a user.

Turning now to FIG. 6, once the pedicle screws are in place, an assembly may be coupled to the pedicle screws. For instance, FIG. 6 shows an example medical implant device 24. More specifically, medical implant device 24 may be a stabilization device that may include pedicle screws (or “anchors”) 26 and 28 that are inserted into vertebrae of a patient's spine, such as vertebrae L4 and L5, respectively. The pedicle screws have off-axis bores 30 and 32 which have been used in conjunction with guide wires (not shown) to guide the screws to the proper location. Assemblies 34 and 36 may be coupled to pedicle screws 26 and 28, respectively. Such assemblies 34 and 36 each form a receiving member for receiving closure member (e.g., set screw 38 or 40).

As further shown in FIG. 6, a brace (or “rod”) 42 extends from assembly 36 to assembly 34, and closure members (e.g., set screws) 38 and 40 are used for securing a first end of the brace to the pedicle screw 28 and the other end of the brace 42 to pedicle screw 26.

During the installation of the brace 42, the second end of the brace may be inadvertently dropped by the surgeon when it is being placed into the assembly 34. The surgeon will need to retrieve the second end of the brace 42 and place it into the assembly 34 before the surgeon can proceed.

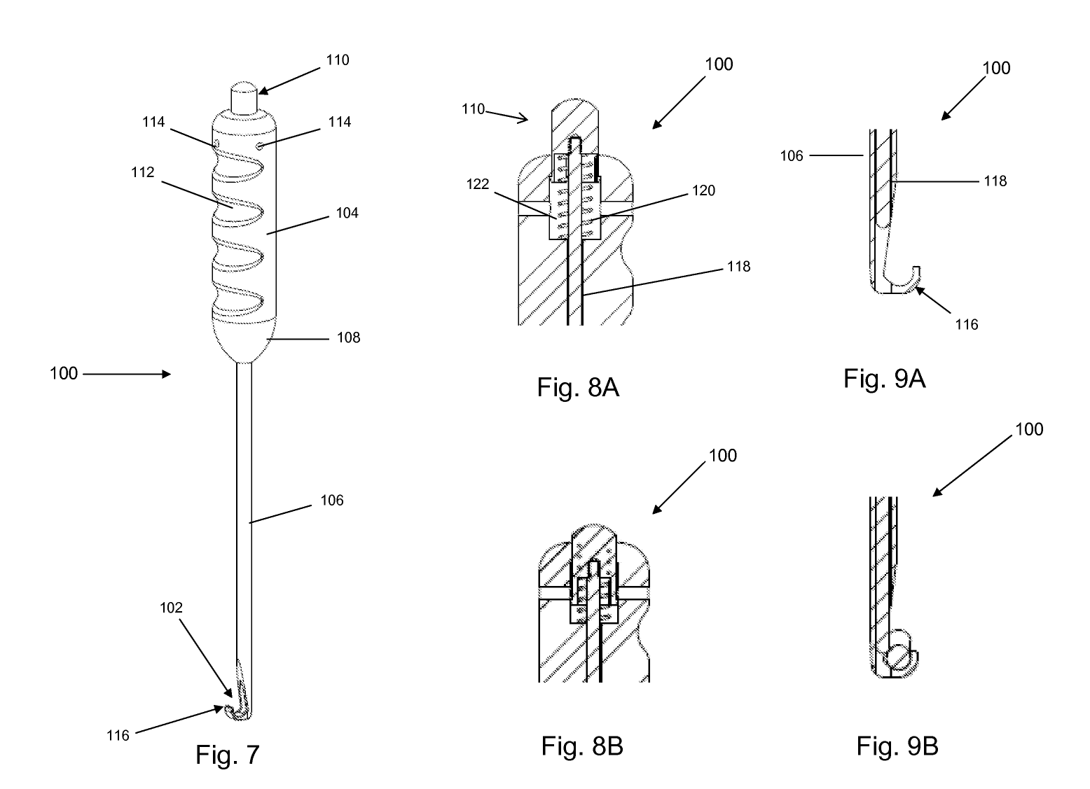

Turning now to FIG. 7, there is illustrated one embodiment of a medical implant holding apparatus 100 that may assist the surgeon in retrieving the second end of the brace 42. In certain embodiments, there may be an implant engaging mechanism 102 located at or close to the distal end. The implant engaging mechanism 102 may be adapted to engage a medical implant, such as the brace 42. The implant engaging mechanism 102 may capture, hold, and/or manipulate the implant during a subcutaneous or percutaneous surgical procedure.

In certain embodiments, there may be a gripping surface, such as a handle 104 which may be located at or close to the proximal end of the apparatus. The handle 104 may be configured so that the apparatus 100 may be operated using only one hand. In some embodiments, the handle 104 may further comprise indentions, such as indentation 112 to facilitate easier gripping of the apparatus 100 by a user thereof. Located near the proximal end portion of the handle 104 there may be orifices 114 for cleaning of the apparatus 100 following use during a surgical procedure. (A vacuum source may be used to suction out any tissue, fluids, and any other contaminants that may collect inside the apparatus during a surgical procedure.)

The apparatus may include a tubular member or sleeve 106 extending from the distal portion 108 of the handle 104 to the implant engaging mechanism 102. The sleeve 106 and the implant engaging mechanism 102 may be relatively sized and proportioned with respect to each other to enable a user of the apparatus to maintain visual contact with a medical implant during capture and manipulation thereof.

Additionally, in some embodiments, the holding apparatus 100 may include an actuator 110, which may partially comprise a pusher cap, for actuating the implant engaging mechanism 102. In some embodiments, the actuator 110 may be located close to or at the proximal end of the apparatus 100. In certain embodiments, the actuator 110 may be slidably positioned within the handle 104 such that the actuator 110 is capable of sliding between a first position and a second position.

An implant engaging shape, such as a hook 116 may be disposed at the distal end of the sleeve 106. In certain embodiments, the hook 116 may be secured onto a distal end of the sleeve 106 by an adhesive, weldment, or other suitable substantially permanent fastening means known to those in the art of manufacturing surgical instruments. In other embodiments, the hook 116 may be formed directly onto the distal end of the sleeve 106.

Referring to FIG. 8A, there is shown a sectional view of the proximal end of the apparatus 100 in an unactuated state or unengaged position. As illustrated, the pusher cap or actuator 110 and a pusher stem 118 located within a bore 122 are in a first or unengaged position. When the pusher stem 118 is not actuated and resting in the first position, the proximal end of the pusher stem 118 may rest within the actuator 110 and within an uncompressed spring 120 that surrounds the pusher stem 118. In certain embodiments, the pusher stem 118 may be threadably coupled to the actuator 110 at its proximal end.

Turning now to FIG. 9A, there is shown a detailed section view of the distal end of the apparatus 100 corresponding to unactuated state of the proximal end of the apparatus 100 shown in FIG. 8A. Thus, when the pusher stem 118 is in the first position, the stem 118 is in an unengaged or “retracted” position with respect to the sleeve 106 and the hook 116.

Referring to FIG. 8B, when the actuator 110 is pressed downward, the actuator 110 moves the pusher stem 118 towards the distal end and compresses the spring 120. When the actuator 110 is pressed downward and the spring 120 is compressed, the pusher stem 118 is moved downward toward the hook 116 and into the second position to capture and hold a medical implant between the distal end of the stem 118 and the hook 116, as illustrated in FIG. 9B. In some embodiments, the stem 118 may be coupled inside the actuator cap with the spring surrounding the proximal end of the stem 118. The proximal end of the stem 118 may be coupled with the actuator 110 via an adhesive, a weldment, or other substantially permanent fastening means known to those in the art of manufacturing medical instruments. In other embodiments, the actuator 110 and the proximal end of the stem 118 may be threaded such that the stem 118 threadedly engages the actuator 110.

Focusing now on FIG. 9B, as the pusher stem 118 is pressed downward toward the hook 116, an implant or rod S may be engaged by the pusher stem 118. The implant S, therefore is held in place between the pusher stem 118 and the hook 116. Once held between the stem 118 and the hook 116, the implant S may rotate freely, enabling a user of the apparatus 100 to change the position of the apparatus 100 as needed for visibility purposes. To release the hold of the implant S, a user may push down on the actuator 110 releasing the lips from a rim and the spring 120 uncompresses thereby propelling the stem 26 back to the first position.

As shown in FIG. 9B, when the stem 118 is actuated and in the second position, the distal end of the stem 118 extends outwardly from the sleeve 106 toward the hook 116 thereby engaging and holding a spinal rod S between the distal end of the stem 118 and the hook 116. The hook 116 has a wider profile relative to the sleeve 106 and the rod S. The difference in the profiles of the sleeve and the hook are represented by example width W. In some embodiments, the diameter of the sleeve 106 may be smaller than the diameter of the spinal rod S or similarly sized implant such that the sleeve does not extend over the implant captured in the hook, thereby enabling a user of the apparatus 100 to maintain visibility of the spinal rod S during and after capture by the apparatus 100.

To retrieve an implant using the apparatus 100, a user engages and captures an implant with the hook 116. The user then presses the cap 110 downward to engage the spring 120 and actuate the stem 118 toward the second position. The stem 118 engages the implant downward into the hook 116 and the implant is thereafter held between the distal end of the pusher stem 118 and the hook 116. Accordingly, the minimum amount of pressure required to depress the cap 110 may be similar to the pressure required to operate a retractable pen or the like.

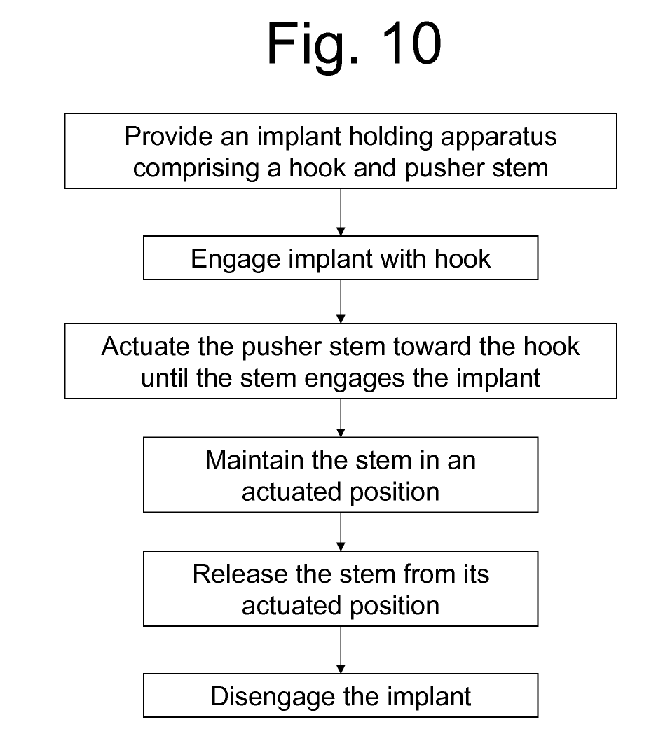

Referring to FIG. 10, there is shown a flowchart illustrating one example of a method of retrieving a medical implant using the apparatus 100. During a surgical procedure, a surgeon may desire to capture a medical implant for manipulation into a bone anchor such as a pedicle screw, or may desire to capture a misplaced medical implant for further manipulation into a spinal stabilization system. A surgeon may use a medical implant grabbing apparatus such as the apparatus 100 described herein for retrieval and capture of the medical implant. Holding the apparatus by a handle, once the surgeon locates the medical implant, the surgeon then engages the medical implant with a hook-like mechanism maintaining visual contact with the implant. The surgeon thereafter presses a cap or button-like mechanism of a spring-loaded plunger system thereby compressing a spring and actuating a pusher stem coupled to the spring. Accordingly, the minimum amount of pressure required to depress the cap or button-like mechanism may be similar to the pressure required to operate a retractable pen or the like thereby enabling the surgeon to actuate the pusher stem with minimal effort or use of a second hand or any other aids. The spring compresses and the pusher stem is pushed toward the medical implant until the distal end of the stem engages the implant and the implant is thereafter captured between the stem and the hook. Tabs of the cap having lips on the perimeters thereof engage an inner lip within the handle to retain the cap in a depressed state, maintaining the spring compressed and the pusher stem in mating engagement with the medical implant. The medical implant is thereafter held between the hook and the pusher stem until the cap or button-like mechanism is depressed again to release the spring and disengage the stem from the implant. Once the surgeon has placed the implant or desires to release the implant, the surgeon depresses the cap or button-like mechanism releasing the stem from engagement with the implant thereby releasing the apparatus's hold of the implant.

After the apparatus 100 has successfully moved the misplaced rod 42 or implant S, the apparatus 100 is removed from the patient and the in progress surgical procedure may continue.

During the surgical procedure, the extension 22 should remain coupled to each pedicle screw (26 and 28) until the brace 42 is secured by closure members 38 and 40, and an access path that the extension 22 may provide is no longer needed by the surgeon.

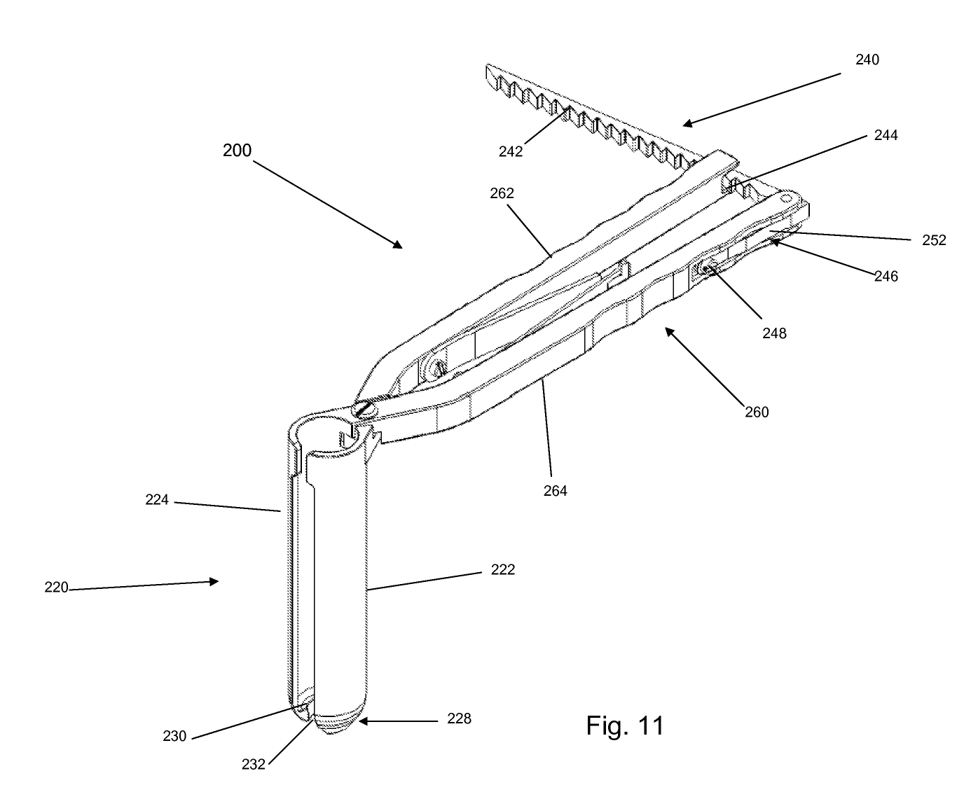

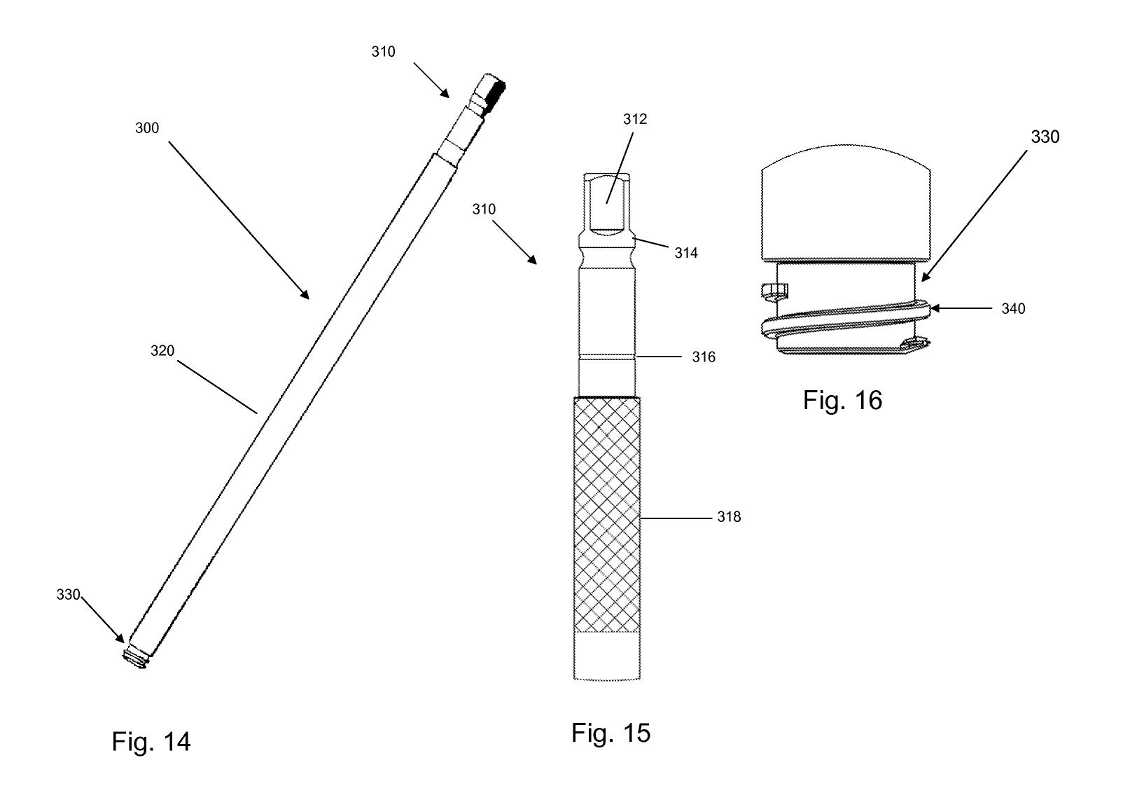

An extension 22 is coupled to an implant assembly or poly-axial head assembly being installed into a vertebra. The distal end of the extension 22 may become separated or uncoupled from the poly-axial head. A surgeon may then want to re-grasp the poly-axial head and reconnect the extension 22 to continue the surgical procedure. The poly-axial head may not be in a stable or locked position, thus the angle of the poly-axial head may change relative to the pedicle screw. Since, the poly-axial head is not fixed in place; it may be difficult for the surgeon to re-connect the extension 22 without utilizing invasive surgical instruments or even grasping the poly-axial head by hand. The devices of FIG. 11 and 14, the retrieval tube 200 and head stabilizer 300, respectively, may be used by a surgeon to hold the poly-axial head still while the extension 22 is reattached.

Turning now to FIG. 11, there is presented one illustrated embodiment of a retrieval tube 200 showing certain aspects of the invention. As shown, the retrieval tube 200 may include an implant engaging mechanism 220, a locking mechanism 240 and a handle or grip portion 260. The implant engaging mechanism 220 may include a first tubular portion 222 and a second tubular portion 224. Each tubular portion has a tapered region 228 near the distal end in order to help retract some tissue that surrounds an implant assembly. The handle or grip portion 260 may include a first and second gripping arm 262, 264 and a first and second spring 266, 268.

The locking mechanism 240 includes a ratchet portion 242, a latch 244 and a spring portion 246. The ratchet portion 242 of the device is biased towards the latch 244 by the spring portion 246. The locking mechanism 240 is attached to an arm of the grip portion by a fastener 248 and a pivot 250. The ratchet portion 242 is capable of pivoting about the pivot 250 when a user presses the center portion 252 of the spring portion 246 and thus releasing the bias on the ratchet portion 242 enabling the latch 244 to move along the ratchet portion 242.

The first tubular portion 222 and second tubular portion 224 are capable of moving relative to one another by pivoting around pivot 226. The distal end portion or tapered region 228 of the first and second tubular portions 222, 224 may include undercuts 230 shaped to grip the outside perimeter of an implant when the first and second tubular portions 222, 224 are in a configuration such that the gap 232 between the portions 222, 224 is minimized.

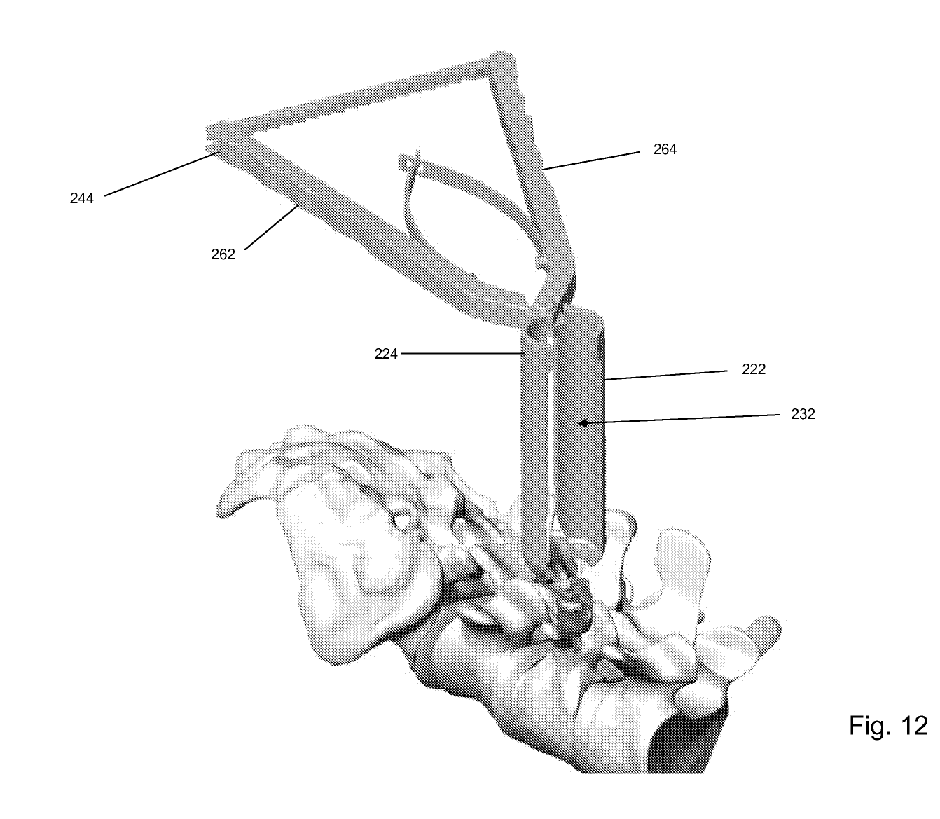

FIGS. 12 and 11 illustrate a first and second position, respectively, of one illustrated embodiment of the present invention. In the first position, the gap 232 between the first and second tubular portions 222, 224 is maximized such that the tubular portions are farther apart and the latch 244 and the first gripping arm 262 are locked in a position farther from the second gripping arm 264. The device may transition from the first position to the second position by a user pressing the center portion 252 (FIG. 11) of the spring portion 246 and moving the first gripping arm 262 and latch 244 towards the second gripping arm 264. In the second position, the gap 232 between the first and second tubular portions 222, 224 is minimized such that the tubular portions are closer together and the latch 244 and the first gripping arm 262 are locked in a position closer to the second gripping arm 264. The second position of the device may vary along the length of the ratchet portion 242.

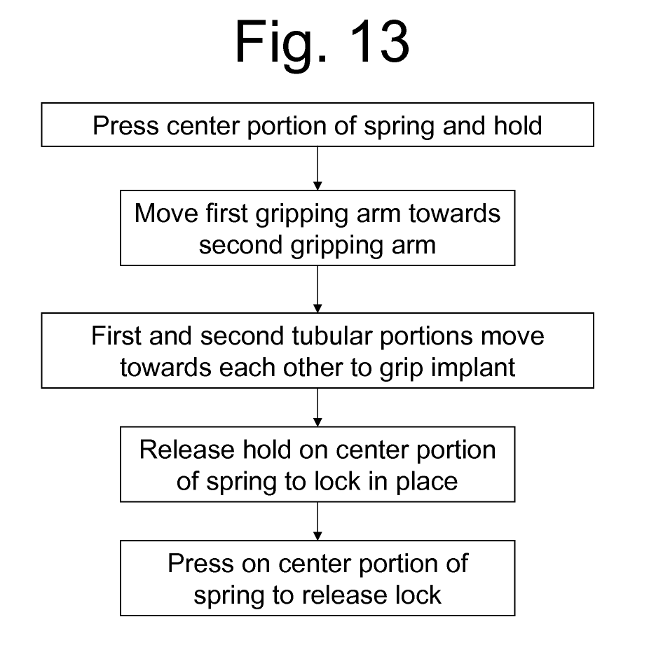

Referring now to FIG. 13, the manner of operating or using one embodiment of the present invention will now be described.

The retrieval tube 200 is opened and locked in the first or open position (FIG. 12). In the open position, the first and second tubular portions 222, 224 have a larger gap 232 between their edges allowing both portions to fit over the exterior of the poly-axial head 210. The user unlocks the retrieval tube 200 and moves the first gripping arm 262 towards the second gripping arm 264 by pressing on the center portion 252 of the spring portion 246. Once the undercuts 230 are gripping the exterior surface of the poly-axial head assembly, the user may release the center portion 252 of the spring portion 252 and lock the first and second tubular portions 222, 224 in place. The extension 22 may then be placed through the lumen created by the first and second tubular portions 222, 224 and reconnected to the projections on the poly-axial head assembly. After the extension 22 is reconnected, the retrieval tube 200 may be removed by pressing on the center portion 252 of the spring portion 246 and moving the first gripping arm 262 away from the second gripping arm 264, thus releasing the grip of undercuts 230 on the poly-axial head assembly.

After the retrieval tube 200 has successfully allowed the re-coupling of the extension to the poly-axial head assembly and is removed from the patient, the in progress surgical procedure may continue.

Turning now to FIG. 14, there is presented one illustrated embodiment of a head stabilizer 300 showing certain aspects of the invention. As shown, the head stabilizer 300 may include a handle interface portion 310, an elongated shaft 320 and an implant engaging mechanism 330.

FIG. 15 is an exploded view of the handle interface portion 310. Certain embodiments of the handle interface portion may include flats 312, top 314, and ring 316. The handle interface portion 310 is capable of mating with a handle so that the user may apply an actuating force to the implant engaging mechanism 330. The handle interface portion 310 also may include a knurled portion 318 in order to allow a user to apply the actuating force by hand.

FIG. 16 shows an exploded view of an implant engaging mechanism 330 that may be used with certain embodiments of this invention. In certain embodiments the implant engaging mechanism 330 may include a helical male thread form or threaded portion 340 that is complementary to the helical female thread form that is located inside a poly-axial head. The thread form on the implant engaging mechanism may include dovetail shapes. Other thread forms are contemplated and are further described in co-pending and commonly assigned U.S. patent application Ser. No. 10/805,967 entitled “Closure Member for a Medical Implant Device,” herein incorporated by reference.

The manner of using one embodiment of the present invention will now be described. A distal end of an extension 22 is attached to a poly-axial head assembly. The distal end of the extension 22 may become separated from the poly-axial head prematurely. A surgeon may then want to re-grasp the poly-axial head and reconnect the extension to continue the surgical procedure. The poly-axial head assembly may not be in a stable or locked position, the angle of the poly-axial head may change relative to the pedicle screw. Since, the poly-axial head is not fixed in place; it may be difficult for the surgeon to re-connect the extension without utilizing invasive surgical instruments or even grasping the poly-axial head by hand. The device of FIG. 14, the head stabilizer 300, may be used by a surgeon to hold the poly-axial head assembly still while the extension 22 is reattached.

The threaded portion 340 of the head stabilizer 300 is threaded into a threaded portion of the poly-axial head. The surgeon may hold a handle attached to the handle interface portion 310 or knurled portion 318 to stabilize the angle of the poly-axial head, making it easier for the extension 22 to be reconnected. In addition, the dimensions of the head stabilizer 300 also may help to minimize damage to the surrounding tissues. After the head stabilizer 300 is connected to the poly-axial head; the extension 22 is slid down over the shaft 320 of the head stabilizer 300. The distal end of the extension 22 is re-connected to the poly-axial head. The threaded portion 340 of the head stabilizer 300 is unthreaded from the poly-axial head assembly and removed from within the extension 22. The surgeon may continue with the surgical procedure that was in progress when the extension 22 was inadvertently disconnected.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

What is claimed is:

1. A kit for use during stabilization procedures comprising:
a head stabilizer having an elongated shaft having a distal end portion and a proximal end portion, wherein an implant engaging portion is coupled to the distal end portion;
a holding apparatus having a sleeve having a proximal end portion and a distal end portion, a pusher shaft received within the sleeve, the pusher shaft having a proximal end portion and a distal end portion, and a first implant engaging mechanism coupled to the distal end portion of the sleeve; and
a retrieval tube having a handle portion having a proximal end portion and a distal end portion, a second implant engaging mechanism coupled to the distal end portion of the handle portion, a locking mechanism coupled to the proximal end portion of the handle portion.

2. The kit of claim 1 wherein the implant engaging portion of the head stabilizer comprises a threaded portion.

3. The kit of claim 1 wherein the elongated shaft of the head stabilizer further comprises a knurled portion.

4. The kit of claim 1 wherein the head stabilizer further comprises a handle interface portion coupled to the proximal end of the elongated shaft.

5. The kit of claim 1 wherein the first implant engaging mechanism of the holding apparatus comprises a hook.

6. The kit of claim 1 wherein the holding apparatus further comprises:
a grip portion having a distal end portion, wherein the grip portion is coupled to the proximal end portion of the sleeve and the grip portion and the sleeve are longitudinally aligned.

7. The kit of claim 6 wherein the grip portion of the holding apparatus further comprises orifices extending there through.

8. The kit of claim 6 wherein the holding apparatus further comprises:
an actuator cap coupled to the proximal end portion of the pusher shaft and the proximal end portion of the grip portion.

9. The kit of claim 1 wherein the head stabilizer, holding apparatus, and retrieval tube are included in a variety of sizes.

10. The kit of claim 1 wherein the locking mechanism of the retrieval tube is a ratchet.

11. The kit of claim 1 wherein the implant engaging mechanism comprises a first tubular portion having a first distal end portion and a second tubular portion having a second distal end portion; wherein the first and second distal end portions have a tapered region and an undercut.

12. The kit of claim 1 further comprising:
a guide wire;
a plurality of pedicle anchors;
a plurality of dilators,
a plurality of extensions adapted to be coupled to the plurality of pedicle anchors;
a plurality of poly-axial heads adapted to be coupled to a pedicle anchor; and
a plurality of spinal rods adapted to couple at least a pair of pedicle anchors together.

13. A method of using a kit for stabilization procedures, comprising:
implanting a spinal stabilization device, wherein the spinal stabilization device comprises a pair of pedicle screws, each screw coupled to a poly-axial head, and a connecting rod;
positioning an implant engaging mechanism of a holding apparatus around the connecting rod;
actuating the implant engaging mechanism to hold the connecting rod;
locking the implant engaging mechanism against the connecting rod to hold the rod; and
manipulating the position of the connecting rod by moving the holding apparatus.

14. The method of claim 13 further comprising:
unlocking the implant engaging mechanism and releasing the hold on the connecting rod.

15. The method of claim 13 wherein the actuating step and locking step are one step wherein an actuator cap of the holding apparatus is pressed and locked, wherein the cap comprises a plurality of tabs having outwardly extending lips and wherein the actuator cap is coupled to a biased spring.

16. The method of claim 13 further comprising:
selecting a retrieval tube having first and second tubular portions having a gripping portion, a first arm and a second arm, and a biased locking mechanism;
placing the first and second tubular portion of a retrieval tube around an implanted poly-axial head;
releasing the bias on the locking mechanism;
moving the first arm of the retrieval arm towards the second arm of the retrieval tube after releasing the bias on the locking mechanism;
gripping the poly-axial head with the gripping portion of the retrieval tube; and
locking the retrieval tube by locking the position of the first arm relative to the second arm.

17. The method of claim 16 further comprising:
placing an extension through the first and second tubular portions;
coupling the extension to the poly-axial head gripped by the retrieval tube; and
releasing the poly-axial head from the gripping portion of the retrieval tube after coupling the extension.

18. The method of claim 13 further comprising:
selecting a head stabilizer having an implant engaging portion;
coupling the implant engaging portion to one of the implanted poly-axial heads.

19. The method of claim 18 further comprising:
sliding an extension over the length of the head stabilizer;
coupling the extension to the poly-axial head; and
uncouple the head stabilizer from the implanted poly-axial head after coupling the extension to the poly-axial head.
